(19) United States
(12) Patent Application Publication
(10) Pub. No.: US 2026/0102580 A1
(43) Pub. Date: Apr. 16, 2026

(54) NOVEL PRESSURE-CONTROLLED EXHALE RELIEF VALVE

(71) Applicant: Derrick Jack, Mesa, AZ (US)

(72) Inventor: Derrick Jack, Mesa, AZ (US)

(21) Appl. No.: 18/915,814

(22) Filed: Oct. 15, 2024

(51) Int. Cl. A61M 16/20; A61M 16/06

(52) U.S. Cl. CPC A61M 16/201; A61M 16/0605

(57) ABSTRACT

A dual pressure zone PAP mask is provided. The dual pressure zone PAP mask includes a nasal mask coupled to a PAP air supply with a PAP airline, an oral mask, and a pressure relief vent valve. The pressure relief vent valve may be coupled between the PAP airline and the oral mask. The pressure relief vent valve may be configured to move between an open and a shut position to allow exhalation through the oral mask in response to a pressure in the oral mask exceeding a pressure in the PAP airline. A respiratory therapy method is also provided through utilization of the dual pressure zone PAP mask and pressure relief vent valve to address exhalation blockage attributed to Expiratory Obstructive Sleep Apnea and/or Expiratory Flow Limitation.

### BACKGROUND OF THE INVENTION

### Technical Field

This invention relates generally to positive airway pressure masks and more particularly to a dual pressure zone positive airway pressure mask.

### State of the Art

Expiratory Obstructive Sleep Apnea and Expiratory Flow Limitation (“EFL”) are respiratory disorders characterized by an individual's inability to exhale smoothly through the nose due to periodic floppy tissue collapse in the nasal airway that restricts or blocks airflow during exhalation. Individuals with this condition may or may not also experience classic Obstructive Sleep Apnea (“OSA”).

Afflicted individuals are resistant to existing apnea therapeutics such as: Positive Airway Pressure (“PAP”), Continuous Positive Airway Pressure (“CPAP”), Automatic Positive Airway Pressure (“APAP”), Bi-Level PAP, Expiratory Positive Airway Pressure (“EPAP”), Sleep Apnea Oral Appliances, and other respiratory therapies and techniques because none address floppy tissue in the airway that can cause an exhalation blockage. Surgery is currently the only viable option for relief.

The current medical best-practice is to prescribe PAP therapy using a full-face PAP mask. Full-face PAP masks flow PAP air pressure equally to both nose and mouth so nasal and oral breathing are equally possible. Whenever the nasal passage is restricted or blocked, the mouth opens, and mouth breathing begins. Mouth breathing will continue for the remainder of the sleep period unless the nasal passage is cleared by a swallow or muscle gag reflex when the sleeper is sufficiently roused. Mouth breathing tends to replace nasal breathing because the path to the lungs is shorter and less tortuous from the mouth than from the nose. Restoration of nasal breathing is not likely in a full-face mask, especially for those afflicted with exhalation blockage related apnea. Full-face masks do nothing to help clear nasal exhalation obstruction and therefore are incapable of promoting healthy nasal breathing during sleep. This becomes a problem for the user because regular mouth breathing is associated with many health issues including deteriorating nasal passage strength, worsening nasal congestion, increased risk of respiratory/nasal infection, dental decay, some heart conditions, and so forth.

Accordingly, there is a need for a novel dual pressure zone positive airway pressure mask.

### SUMMARY OF THE INVENTION

An embodiment includes a dual pressure zone PAP mask comprising: a nasal mask configured to interface with a nose of a user, the nasal mask coupled to a PAP airline; an oral mask configured to interface with a mouth of the user; and a pressure relief vent valve, wherein: the pressure relief vent valve is coupled between the PAP airline and the oral mask; the pressure relief vent valve is configured to move from a shut position and an open position to allow exhalation through the oral mask in response to a pressure in the oral mask exceeding a pressure in the PAP airline by a predetermined value; and the pressure relief vent valve is configured to move from the open position to the closed position to prevent exhalation through the oral mask in response to the pressure in the oral mask being less than the pressure in the PAP airline by the predetermined value.

Another embodiment includes a pressure relief vent valve for use with a dual pressure zone PAP mask, the pressure relief vent valve comprising: an inner volume; an inlet port extending into the inner volume; an oral relief port extending into the inner volume; a vent port extending into the inner volume; and a piston/valve assembly operably coupled within the inner volume, wherein the piston/valve assembly is moveable between a position engaged with a valve seat of the oral relief port to inhibit air flow and a position disengaged with the oral relief port to allow air flow.

Another embodiment includes a respiratory therapy method comprising: coupling a dual pressure zone PAP mask to a user, wherein the dual pressure zone PAP mask comprises: a nasal mask configured to interface with a nose of the user, the nasal mask coupled to a PAP airline; an oral mask configured to interface with a mouth of the user; and a pressure relief vent valve, wherein: the pressure relief vent valve is coupled between the PAP airline and the oral mask; the pressure relief vent valve is configured to move from a shut position and an open position to allow exhalation through the oral mask in response to a pressure in the oral mask exceeding a pressure in the PAP airline by a predetermined value; and the pressure relief vent valve is configured to move from the open position to the closed position to prevent exhalation through the oral mask in response to the pressure in the oral mask being less than the pressure in the PAP airline by the predetermined value; performing a nasal inhalation and an unobstructed nasal exhalation through the nasal mask; in response to an airway of the user being obstructed by a blocking tissue of the user, perform an obstructed exhalation, wherein the blocked nasal passage creates oral air pressure in the oral mask greater than the pressure in the PAP airline by the predetermined value to move the pressure relief vent valve from the shut position to the open position venting the oral exhalation through the oral mask and out a vent port of the pressure relief valve; in response to venting the oral exhalation, drawing in nasal air pressure from the PAP airline to clear the obstruction; pushing the blocking tissue out of the nasal airway as the nasal air pressure from the PAP airline engages the blocking tissue while the oral air pressure drops from venting the oral exhalation; as the oral exhalation ends, moving the pressure relief vent valve from the open position to the shut position in response to the incoming PAP airline pressure exceeding the oral air pressure in the oral mask by the predetermined value; and performing a nasal inhalation and an unobstructed nasal exhalation through the nasal mask.

The foregoing and other features and advantages of the present invention will be apparent from the following more detailed description of the particular embodiments of the invention, as illustrated in the accompanying drawings.

### BRIEF DESCRIPTION OF THE DRAWINGS

A more complete understanding of the present invention may be derived by referring to the detailed description and claims when considered in connection with the Figures, wherein like reference numbers refer to similar items throughout the Figures, and:

FIG. 1 is a side view of one embodiment of a dual pressure zone PAP mask according to an embodiment;

FIG. 2 is a side view of another embodiment of a dual pressure zone PAP mask according to an embodiment;

FIG. 3 is a section view of operation of a dual pressure zone PAP mask with a pressure-controlled exhale relief vent valve shown in a shut position, according to an embodiment;

FIG. 4 is a section view of operation of a dual pressure zone PAP mask with a pressure-controlled exhale relief vent valve shown in a partially open position according to an embodiment;

FIG. 5 is a section view of operation of a dual pressure zone PAP mask with a pressure-controlled exhale relief vent valve shown in an open position for venting according to an embodiment;

FIG. 6 is a section view of the operation of a dual pressure zone PAP mask with a pressure-controlled exhale relief vent valve shown in the shut position following venting according to an embodiment;

FIG. 7 is a view of an inhalation path during a respiratory therapy method using a dual pressure zone PAP mask with a pressure-controlled exhale relief vent valve according to an embodiment;

FIG. 8 is a view of an unobstructed exhalation path during a respiratory therapy method using a dual pressure zone PAP mask with a pressure-controlled exhale relief vent valve according to an embodiment;

FIG. 9 is a view of an obstructed exhalation path during a respiratory therapy method using a dual pressure zone PAP mask with a pressure-controlled exhale relief valve according to an embodiment;

FIG. 10 is a view of an obstructed exhalation path with incoming nasal air pressure during a respiratory therapy method using a dual pressure zone PAP mask with a pressure-controlled exhale relief vent valve according to an embodiment;

FIG. 11 is a section view of the simi-obstructed exhalation path during a respiratory therapy method using a dual pressure zone PAP mask with a pressure-controlled exhale relief vent valve according to an embodiment; and

FIG. 12 is a view of a re-opened unobstructed nasal exhalation path during a respiratory therapy method using a dual pressure zone PAP mask with a pressure-controlled exhale relief vent valve according to an embodiment.

### DETAILED DESCRIPTION OF EMBODIMENTS OF THE INVENTION

As discussed above, embodiments of the present invention relate to a novel dual pressure zone positive airway pressure mask that may include a pressure-controlled exhale relief vent valve. The operation of the dual pressure zone PAP mask with pressure-controlled exhale relief vent valve, performs a respiratory therapeutic method that monitors and manipulates respiratory airflow through a user's nose and mouth via separate oral and nasal pressure zones to automatically detect a pressure differential indicating an exhalation blockage. The dual pressure zone PAP mask automatically takes corrective action by redirecting exhalation airflow away from the oral pressure zone of the mask via an atmospheric vent opened by the pressure-controlled exhale relief vent valve to prevent harmful exhalation pressure buildup while also lowering oral air pressure behind the blockage to a point less than incoming nasal PAP pressure in front of the blockage. Thereby, the dual pressure zone PAP mask draws nasal PAP airflow inward past the blocking tissue, pushing it out of the way, clearing the airway, and then automatically closes the valve once pressure differentials normalize, indicating the blockage is cleared and the airway is open. This restores natural unobstructed nasal respiration within the single obstructed exhalation cycle without disrupting respiratory cadence or the sleep cycle of the user. Use of embodiments restores healthy nasal respiration within the period of a single blocked exhalation and is powered simply by air pressure provided from the PAP hose in the usual way.

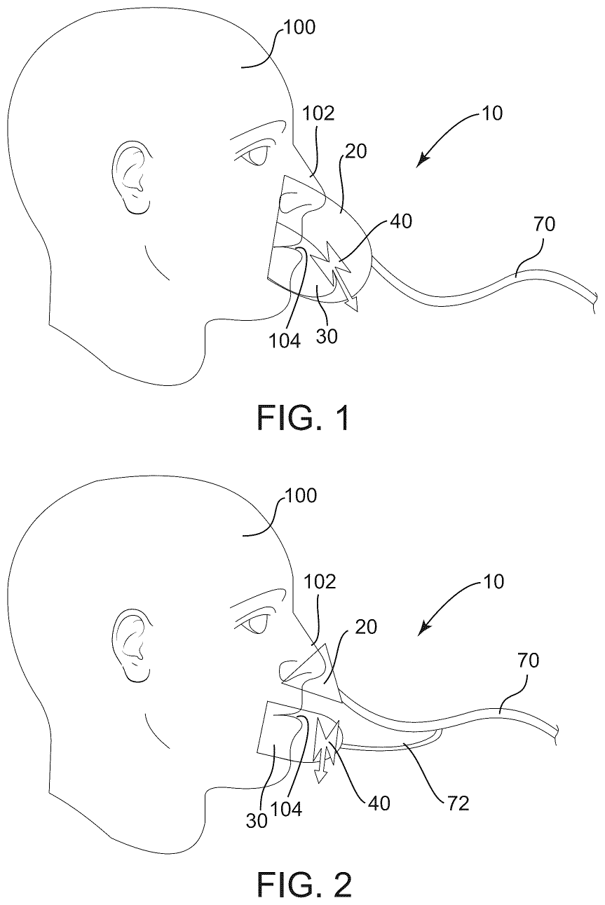

Referring to FIGS. 1 and 2, embodiments of a dual pressure zone PAP mask 10 are depicted. FIG. 1 depicts the dual pressure zone PAP mask 10 configured as a one-piece mask that comprises a nasal pressure zone 20, fed by PAP airline 70, which functions in the usual way as a conventional nasal mask, and an oral pressure zone 30 that is separated from the nasal pressure zone 20 by a pressure barrier comprising a pressure relief vent valve 40. Pressure relief vent valve 40 fed from PAP airline 70, and operates such that PAP airline 70 pressure holds pressure relief vent valve 40 in a shut position until oral exhalation pressure in oral pressure zone 30 rises to a predetermined pressure differential in comparison to pressure in PAP airline 70, causing the pressure relief vent valve 40 to open, venting pressure from oral pressure zone 30 to the atmosphere through a vent path.

FIG. 2 depicts a dual pressure zone PAP mask 10 configured as a two-piece mask comprising a nasal mask 20 fed by PAP airline 70, which functions in the usual way as a conventional nasal mask, and an oral mask 30 coupled to a pressure relief vent valve 40, which is also fed from PAP airline 70. Dual pressure zone PAP mask 10 operates such that PAP airline 70 pressure holds pressure relief vent valve 40 in a shut position until oral exhalation pressure in oral mask 30 rises to a predetermined pressure differential in comparison to pressure in PAP airline 70, causing pressure relief vent valve 40 to open, venting pressure from oral mask 30 to the atmosphere through a vent path.

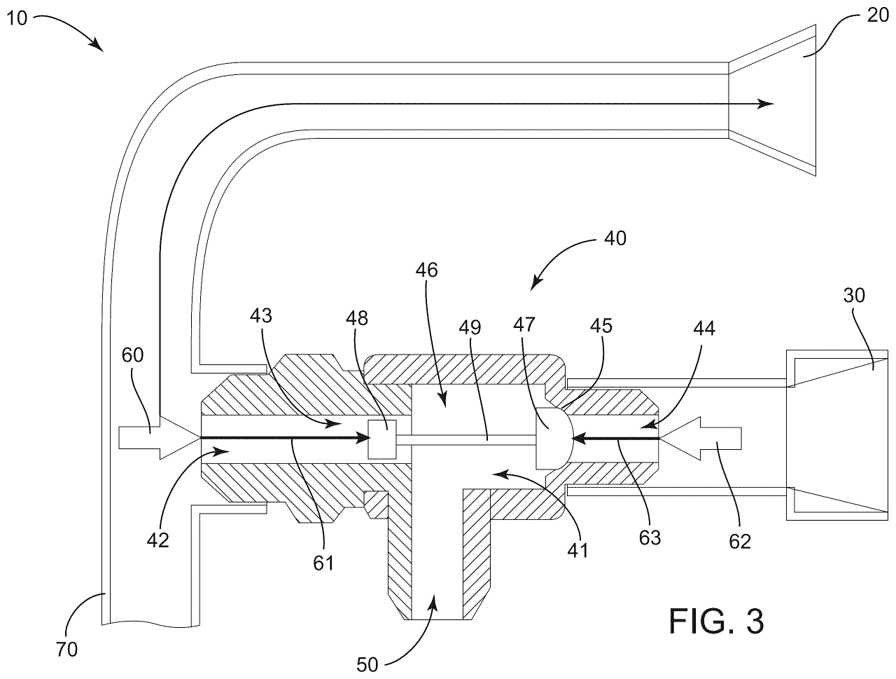

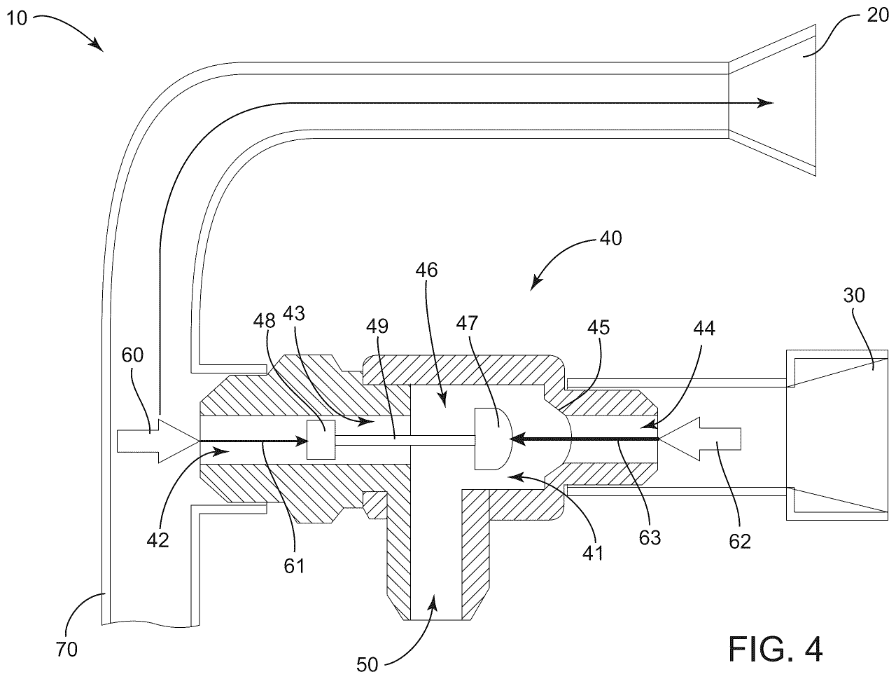

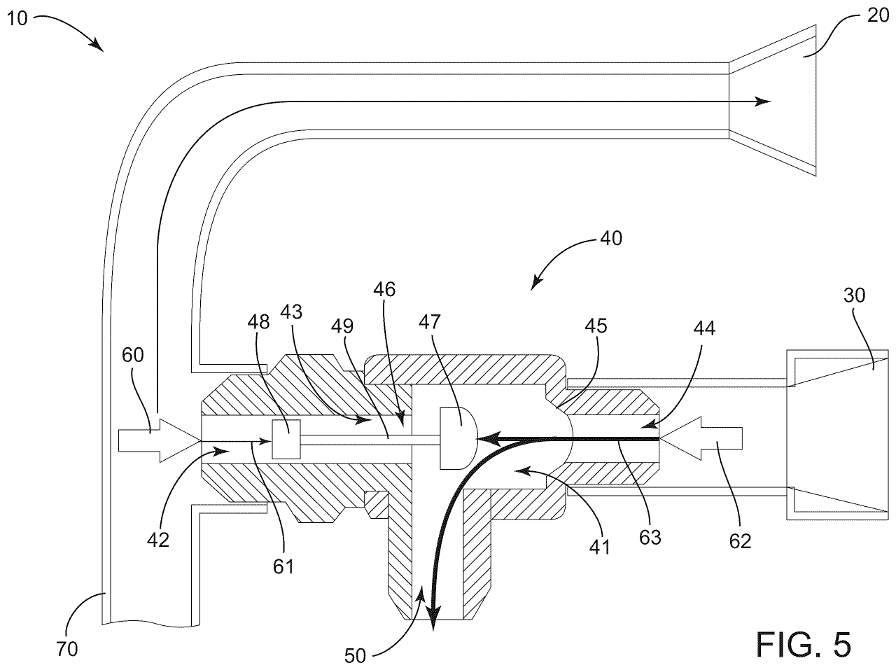

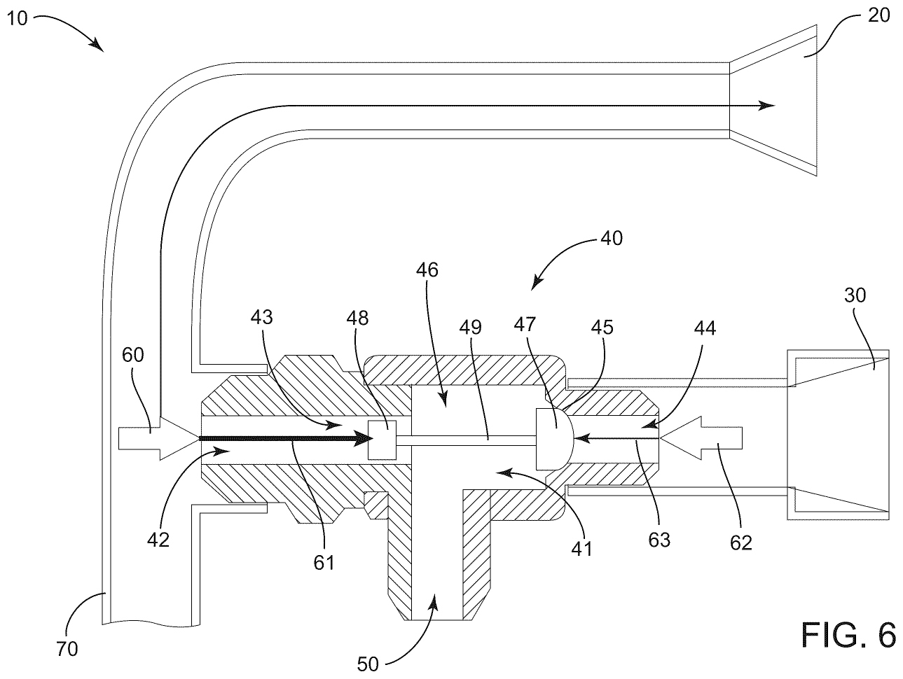

In this disclosure, an embodiment of a two-piece dual pressure zone PAP mask 10 is depicted in FIGS. 3-6 and discussed, however it will be understood that the elements of the two-piece mask can be formed as a one-piece mask having similar components with the same numbering in the drawings and functioning in the same way. Accordingly, dual pressure zone PAP mask 10 comprises a nasal mask 20, an oral mask 30 and a pressure relief vent valve 40. Pressure relief vent valve 40 comprises an inner volume 41, an inlet port 42 extending through cylinder 43 to inner volume 41, an oral relief port 44 extending into inner volume 41 and terminating internally at valve seat 45, and a vent port 50 extending from the atmosphere into inner volume 41. Pressure relief vent valve 40 further comprises a piston/valve assembly 46 that comprises a valve disk 47, a piston 48 with a linkage 49 extending between the valve disk 47 and the piston 48. Piston 48 is located in cylinder 43 extending from or formed as part of inlet port 42 and is connected via linkage 49 to valve disk 47, which is located within the inner volume 41 and in a position to engage and disengage with valve seat 45. It will be understood that alignment guides not depicted in the drawing may be located within inner volume 41 to align piston/valve assembly 46 such that it is located on a center axis formed by inlet port 42, piston cylinder 43, valve seat 45, and oral relief port 43, and is constrained such that piston/valve assembly 46 only translates a small distance axially when piston 48 moves inside cylinder 43 and valve disk 47 moves such that it engages and disengages valve seat 45. Piston/valve assembly 46 is configured to move between a shut position and an open position. In the shut position, valve disk 47 is engaged with valve seat 45 of oral relief port 44 preventing air flow through the oral relief port 44. In the open position valve disk 47 is disengaged from valve seat 45 of relief valve port 44 allowing air flow through the oral relief port 44 and through vent port 50 into the atmosphere. Again, piston 48 is configured to move within cylinder 43 of inlet port 42, wherein linkage 49 coupled between piston 48 and valve disk 47 translates movement of piston 48 within cylinder 43 to movement of valve disk 47 between engagement with and disengagement from valve seat 45.

Nasal mask 20 is coupled to PAP airline 70 extending from a PAP air source (not shown), to provide PAP air pressure 60 for nasal respiration in the usual way. Oral mask 30 is coupled to pressure relief vent valve 40 at oral relief port 44. Pressure relief vent valve 40 is also coupled to a branching airline extending from PAP airline 70 to inlet port 42 to direct PAP air pressure 60 through inlet port 42 and into cylinder 43 where the pressure is applied to piston 48 to piston/valve assembly 46 into the shut position, with valve disk 47 engaging valve seat 45. In some embodiments, a nominal volume of PAP air may escape past piston 48 to inner volume 41 where it can vent to the atmosphere through vent port 50. It will be understood that elements of various components of the present invention, such as, but not limited to, piston 48 diameter, cylinder 43 diameter, and valve seat 44 diameter may be varied to establish an exhalation resistance value for a particular pressure relief vent valve 40 design. Pressure relief vent valve 40 may be produced as an interchangeable subcomponent of dual pressure zone PAP mask 10 enabling customization of user experience by providing multiple pressure relief vent valves 40 covering a range of exhalation resistance values. In these embodiments, a technician or user may choose a valve with a specific resistive valve to optimize therapeutic benefit and/or comfort specific to the user. Further, in some embodiments, pressure relief vent valves 40 may comprise an electronic encoder, such as, but not limited to, an electronic encoder located within inner volume 41, wherein the electronic encoder signals when piston/valve assembly 46 cycles between open and shut positions, thereby aiding sleep study technicians in collecting data on exhalation blockage events during a sleep study. These embodiments may help the technician in determining the optimal resistive value to best benefit an individual patient/user.

FIGS. 3-6 also depict the function of the pressure relief vent valve 40 when coupled with dual pressure zone PAP mask 10, according to an embodiment. Nasal mask 20 is configured to couple to a nose of a user forming a seal with the nose, thereby allowing PAP air pressure 60 to flow through nasal mask 20 and into the nose to function as a nasal airway stent. Oral mask 30 is configured to couple to a mouth of the user forming a seal with the mouth. During unobstructed respiration both inhalation and exhalation gases flow through the nasal airway and nasal mask 20 in the usual way, and pressure relief vent valve 40 remains in the shut position preventing exhalation pressure 62 from flowing past valve seat 45 to prevent oral respiration. When an obstruction prevents exhalation through the nasal passage, exhalation pressure 62 rises in oral mask 30 until the forces 63 acting on valve disk 47 exceed the forces 61 acting against piston 48, thereby pushing piston/valve assembly 46 away from valve seat 45 to an open position to allow venting exhalation volume/pressure 62 through inner volume 41 and out to the atmosphere through vent port 50. This reduces exhalation pressure 62 in oral mask 30 and reduces exhalation pressure 62 below the tissue blockage to allow PAP air pressure 60 above the blockage to flow down from the nose, past the tissue blockage to reopen the nasal airway as nasal PAP air pressure 60 flows down into oral mask 30 to vent with exhalation volume/pressure 62 to the atmosphere. This in turn lowers the pressure in oral mask 30 below PAP air pressure 60 and results in PAP air pressure 60 to apply force 61 to piston 48 to move piston/valve assembly 46 back into the shut position, with valve disk 47 engaged with valve seat 45, resulting in restoring nasal respiration through the open nasal airway and nasal mask 20. Pressure relief vent valve 40 will cycle as described above each time an exhalation obstruction occurs to clear the blockage within the period of a single blocked exhalation. It will be understood that while the function of the pressure relief vent valve 40 is depicted as purely mechanical, it can also operate by electronic means using pressure sensors and an electro-mechanical device.

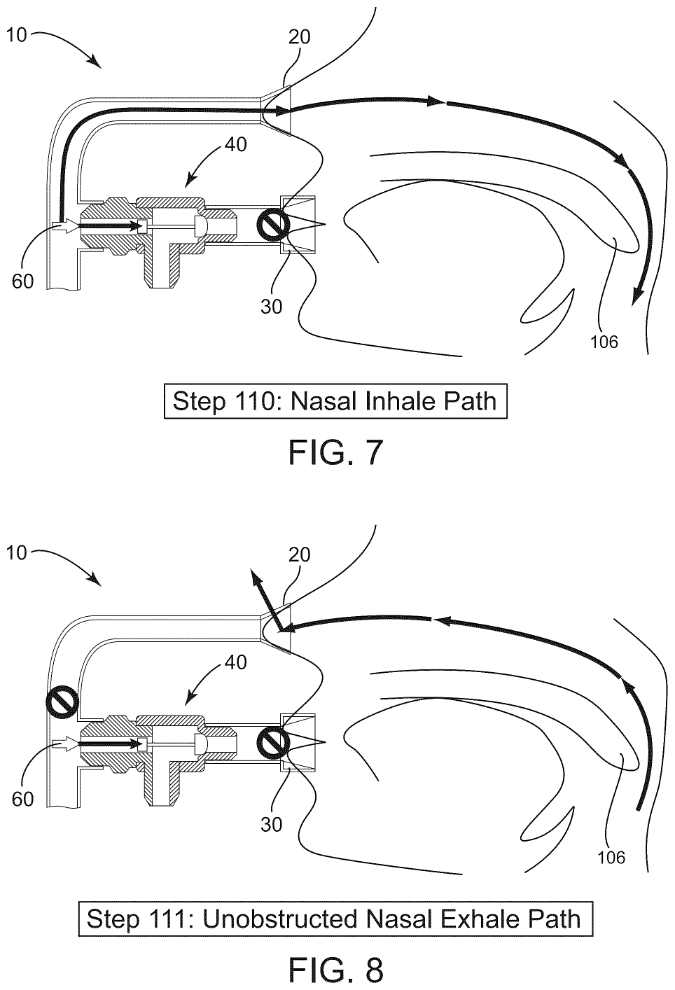

Referring further to FIGS. 7-12 a respiratory therapy method is depicted according to an embodiment. The respiratory therapy method comprises coupling a dual pressure zone PAP mask to a nose and a mouth of a user. The method may further include the following:

Step 110, as shown in FIG. 7, comprises a nasal inhalation that passes smoothly from the nose to the lungs via the nasal airway. In Step 110, the pressure relief vent valve 40 is shut, the nasal passage is clear, and piston/valve assembly 46 is held in the shut position by PAP air pressure 60 acting on piston 48. Inhalation always occurs through the nose as depicted in FIG. 7.

Step 111, as shown in FIG. 8, comprises an unobstructed nasal exhalation that passes smoothly from the lungs to the nose via the nasal airway. In Step 111 pressure relief vent valve 40 is shut, the nasal passage is clear, and piston/valve assembly 46 is held in the shut position by PAP air pressure 60 acting on piston 48. Normal exhalation occurs through the nose when unobstructed. When the nasal passage is unobstructed, exhale through the mouth is inhibited as depicted in FIG. 8.

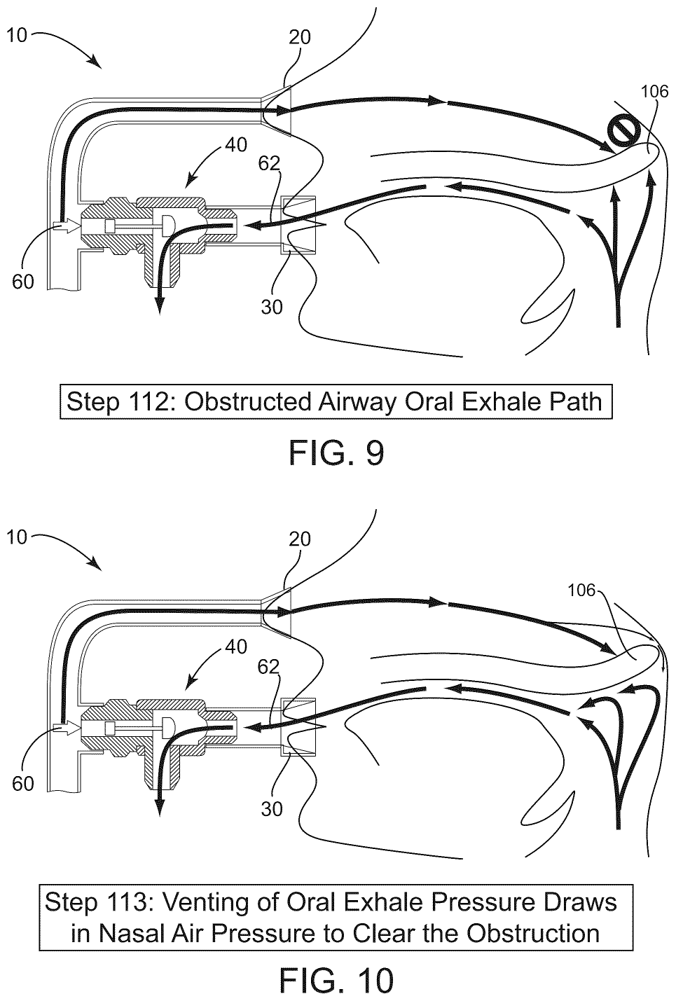

Step 112, as shown in FIG. 9, comprises an obstructed exhalation caused by floppy tissue 106 which has shifted during exhalation, obstructing the nasal airway and preventing exhalation pressure 62 from exiting through the nasal airway. This increases exhalation pressure 62 in oral mask 30 above the exhalation resistance value of pressure relief vent valve 40 and causes pressure relief vent valve 40 to move into the open position and begin venting trapped exhalation pressure 62 to the atmosphere through vent port 50. In Step 112, pressure relief vent valve 40 is starting to open and vent while the nasal airway remains blocked, as depicted in FIG. 9.

Step 113 as shown in FIG. 10, comprises an obstructed exhalation caused by floppy tissue 106 which has shifted during exhalation, obstructing the nasal airway and preventing exhalation pressure 62 from exiting through the nasal airway. This increases exhalation pressure 62 in oral mask 30 above the exhalation resistance value of pressure relief vent valve 40 and causes pressure relief vent valve 40 to move into the open position for venting trapped exhalation pressure 62 to the atmosphere through vent 50. This lowers air pressure below the blockage to a point where pressure from above the blockage can begin pushing past the blocking tissue 106 to free it from the airway. In Step 112, pressure relief vent valve 40 is fully open and venting while the nasal airway remains blocked but the blocking tissue 106 is beginning to be dislodged, as depicted in FIG. 10.

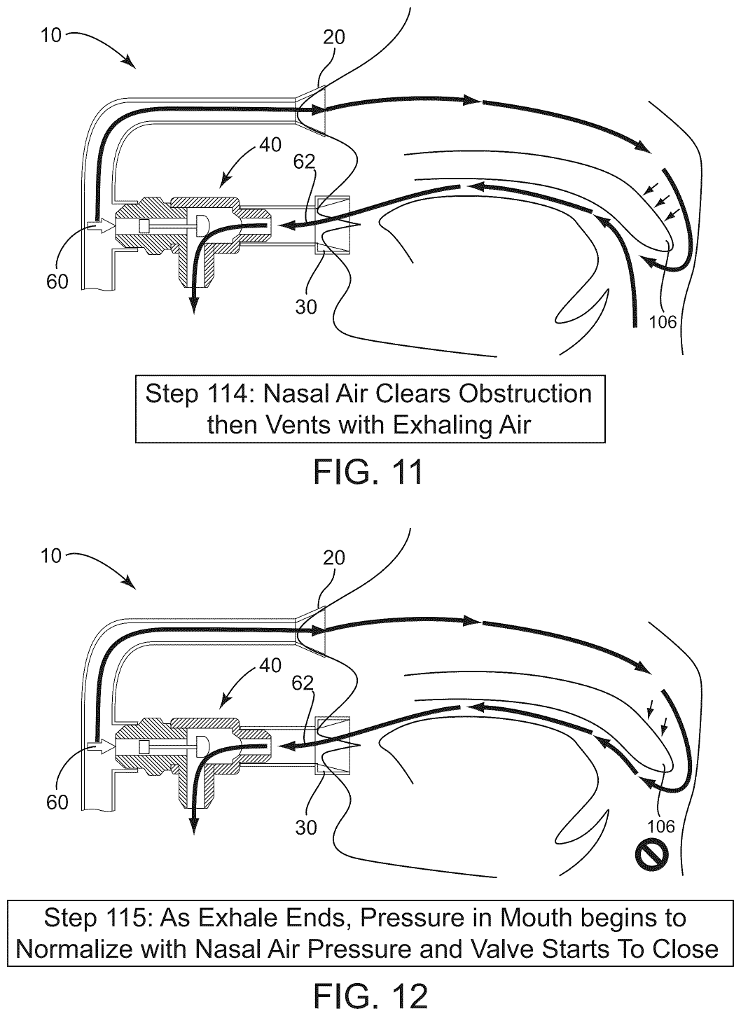

Step 114 as shown in FIG. 11, comprises a partly obstructed nasal airway that is beginning to clear as PAP air pressure 60 supplied from nasal mask 20 rushes down past the blockage; pushing floppy tissue 106 aside opening the airway. The incoming air mixes and vents to the atmosphere with exhalation pressure that continues to flow up from the lungs through the oral mask 30 and out the vent port 50, thereby maintaining pressure relief vent valve 40 in the open venting position. In Step 114, pressure relief vent valve 40 is fully open and venting while the nasal airway begins to reopen as the obstruction is pushed aside, as depicted in FIG. 11.

Step 115 as shown in FIG. 12, comprises a cleared nasal airway with PAP air pressure 60 supplied from nasal mask 20 continuing to rush down past floppy tissue 106, pushing it completely out of the airway as it flows toward pressure relief vent valve 40 that is still in the open position and venting to the atmosphere through vent port 50. As exhalation pressure diminishes to a point where oral air pressure begins to normalize and drop below incoming PAP air pressure 60, the differential in pressure causes pressure relief vent valve 40 to begin closing. In Step 115, pressure relief vent valve 40 starts to close as venting pressure and volume start to diminish and normalize. The nasal airway is no longer obstructed and is ready to resume normal respiratory function, as depicted in FIG. 12.

The respiratory therapy method then starts over as depicted in FIGS. 7 and 8, wherein the respiratory therapy method comprises a cleared nasal airway that has resumed normal nasal respiratory function with no obstruction. As discussed previously with FIGS. 7 and 8 pressure relief vent valve 40 is in the shut position and the nasal airway is open. Normal nasal respiration has resumed and will continue until the next exhalation blockage event, as depicted in FIGS. 7 and 8.

It should be appreciated that some studies suggest one in four breaths will be obstructed, meaning three breaths might occur normally between valve cycles. At first adoption of the dual pressure zone PAP mask 10 and the respiratory therapy method, the user may notice a strange sensation when pressure relief vent valve 40 cycles and causes nasal air to rush in through the nose during expiration, but with continued use the valve cycle and corresponding puff of incoming air will feel normal and be ignored. Over time blocking tissue will become less inflamed further improving the therapeutic benefit from this process. Additionally, over time nasal breathing during sleep will strengthen nasal passage tissue improving nasal respiratory health during both waking and sleeping hours, improving the user's overall health and further decreasing the frequency of blockages during sleep. Over time less frequent blockages will tend to clear faster as nasal tissue is protected from injury by this device. It will be understood that in some cases it may be necessary to provide the user with a mouthpiece designed to facilitate expiratory airflow past the tongue to the oral pressure mask.

Additionally, the embodiments described herein are straightforward, simple, and economical solutions to Obstructive Exhalation Sleep Apnea that can be incorporated using existing respiratory and therapeutic and equipment. Components can be produced using many existing mask materials and similar processes.

This novel PAP mask is intended to help current PAP prescription-holders who struggle to use existing PAP masks and often quit PAP therapy due to poor results. The introduction of this product to the market will tend to attract new customers and bring back former customers willing to try a new option. This novel option will also help retain customers currently dissatisfied and discouraged by poor results from PAP use. An additional benefit of the oral pressure mask as compared to a full-face mask is the comparative ease with which it can seal with a user's mouth because it does not supply PAP airline pressure to the mouth; the mouth is also protected from becoming dry during sleep because no PAP air pressure through the mouth and because air is only permitted to exit the mouth periodically during an exhalation blockage or uncharacteristically heavy exhalation.

This mask may also open prescription PAP use to non-apnea customers with nasal issues and mouth-breathing issues because of the long-term therapeutic health benefits related to nasal airway respiration during sleep. This respiratory process may also be useful in adjacent areas of respiratory medicine such as in respiration during sedation, and in respiratory aids for at-risk infants as a possible risk mitigation aid for contradiction of SIDS.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

1. A dual pressure zone PAP mask comprising:
a nasal mask configured to interface with a nose of a user, the nasal mask coupled to a PAP airline; an oral mask configured to interface with a mouth of the user; and a pressure relief vent valve, wherein: the pressure relief vent valve is coupled between the PAP airline and the oral mask; the pressure relief vent valve is configured to move from a shut position and an open position to allow exhalation through the oral mask in response to a pressure in the oral mask exceeding a pressure in the PAP airline by a predetermined value; and
the pressure relief vent valve is configured to move from the open position to the closed position to prevent exhalation through the oral mask in response to the pressure in the oral mask being less than the pressure in the PAP airline by the predetermined value.

2. The dual pressure zone PAP mask of claim 1, wherein the pressure relief vent valve comprises an inlet port coupled to the PAP airline.

3. The dual pressure zone PAP mask of claim 2, wherein the pressure relief vent valve comprises an oral relief port coupled to the oral mask.

4. The dual pressure zone PAP mask of claim 3, wherein the pressure relief vent valve comprises a vent port, wherein exhalation from the oral mask is directed through the vent port to atmosphere.

5. The dual pressure zone PAP mask of claim 4, wherein the pressure relief vent valve comprises a piston/valve assembly operable within the pressure relief vent valve to move between a position engaged with a valve seat of the oral relief port to inhibit air flow and a position disengaged with the valve seat of the oral relief port to allow air flow.

6. The dual pressure zone PAP mask of claim 5, wherein the open position of the pressure relief vent valve corresponds with the piston/valve assembly in the position disengaged with the valve seat of the oral relief port, and the shut position of the pressure relief vent valve corresponds with the piston/valve assembly in the position engaged with the valve seat of the oral relief port.

7. A pressure relief vent valve for use with a dual pressure zone PAP mask, the pressure relief vent valve comprising:
an inner volume;
an inlet port extending into the inner volume; an oral relief port extending into the inner volume; a vent port extending into the inner volume; and a piston/valve assembly operably coupled within the inner volume, wherein the piston/ valve assembly is moveable between a position engaged with a valve seat of the oral relief port to inhibit air flow and a position disengaged from the valve seat of the oral relief port to allow air flow.

8. The pressure relief vent valve of claim 7, wherein the piston/valve assembly comprises a valve disk and a piston with a linkage extending between the valve disk and the piston.

9. The pressure relief vent valve of claim 8, wherein the piston extends into a cylinder of the inlet port and the linkage locates the valve disk within the inner volume, wherein the valve disk is configured to engage and disengage with the valve seat.

10. The pressure relief vent valve of claim 9, wherein the valve is moveable between a shut position and an open position.

11. The pressure relief vent valve of claim 10, wherein the shut position of the valve comprises the valve disk engaged with the valve seat of the oral relief port preventing air flow through the oral relief port.

12. The pressure relief vent valve of claim 11, wherein the open position of the valve comprises the valve disk disengaged from the valve seat of the oral relief port allowing air flow through the oral relief port.

13. The pressure relief vent valve of claim 12, wherein the piston slides within the cylinder of the inlet port, wherein the piston sliding moves the valve disk between the open and shut positions by use of the linkage between the piston and the valve disk.

14. A respiratory therapy method comprising:
coupling a dual pressure zone PAP mask to a user, wherein the dual pressure zone PAP mask comprises:
a nasal mask configured to interface with a nose of the user, the nasal mask coupled to a PAP airline;
an oral mask configured to interface with a mouth of the user; and
a pressure relief vent valve, wherein:
the pressure relief vent valve is coupled between the PAP airline and the oral mask;
the pressure relief vent valve is configured to move from a shut position and an open position to allow exhalation through the oral mask in response to a pressure in the oral mask exceeding a pressure in the PAP airline by a predetermined value; and
the pressure relief vent valve is configured to move from the open position to the closed position to prevent exhalation through the oral mask in response to the pressure in the oral mask being less than the pressure in the PAP airline by the predetermined value;
performing a nasal inhalation and an unobstructed nasal exhalation through the nasal mask;
in response to an exhalation obstruction of the nasal airway by a blocking tissue of the user during expiration, perform an obstructed oral exhalation, wherein the blocked nasal passage causes an increase in oral air pressure in the oral mask until the oral air pressure if greater than the PAP airline pressure by the predetermined value causing the pressure relief vent valve to move from the shut position to the open position venting the oral exhalation through the oral mask and out a vent port of the pressure relief vent valve;
in response to venting the oral exhalation, drawing in nasal air pressure from the PAP airline to clear the obstruction;
pushing the blocking tissue out of the nasal airway as the nasal air pressure from the PAP airline engages the blocking tissue while the oral air pressure drops from venting the oral exhalation;
as the oral exhalation ends, moving the pressure relief vent valve from the open position to the shut position in response to the incoming PAP airline pressure exceeding the oral air pressure in the oral mask by the predetermined value; and
performing a nasal inhalation and an unobstructed nasal exhalation through the nasal mask.

15. The method of claim 14, further comprising reducing blocking tissue inflammation with continued use of the method over a period of time.

16. The method of claim 15, further comprising strengthening nasal passage tissue with continued use of the method over the period of time.

17. The method of claim 16, further comprising decreasing frequency of blockages and increasing flow rate of nasal air in response to strengthening nasal passage tissue over the period of time.
